(19) United States
(12) Patent Application Publication
(10) Pub. No.: US 2025/0281306 A1
(43) Pub. Date: Sep. 11, 2025

(54) CONVENIENT BONE GRAFT INJECTOR FOR ARTIFICIAL OR ALLOGENEIC BONE

(71) Applicant: The First Affiliated Hospital of Shantou University Medical College, Shantou (CN)

(72) Inventors: Jun HU, Shantou (CN); Shaoke CHEN, Shantou (CN); Jiankun XU, Shantou (CN); Hongjiang CHEN, Shantou (CN); Jiechen CHEN, Shantou (CN)

(73) Assignee: The First Affiliated Hospital of Shantou University Medical College, Shantou, Guangdong (CN)

(21) Appl. No.: 19/056,335

(22) Filed: Feb. 18, 2025

(30) Foreign Application Priority Data
Mar. 5, 2024 (CN) 2024102492324

(51) Int. Cl. A61F 2/46

(52) U.S. Cl. CPC A61F 2/4601

(57) ABSTRACT

A convenient bone graft injector for artificial or allogeneic bone includes: a body, an injection tube, a locking device, and an injection device. The locking device is used to detachably lock one end of the injection tube to the body. The injection device includes a push rod, an injection elastic member, and a locking and releasing mechanism arranged on the body. The injection elastic member acts between one end of the push rod and the body, and the injection elastic member is configured to provide an elastic push to the push rod, such that the other end of the push rod extends into the end of the injection tube locked to the body and moves towards the other end of the injection tube. The locking and releasing mechanism acts between the push rod and the body, and is configured to lock and release the push rod.

### CROSS-REFERENCE TO RELATED APPLICATION

This application is based on and claims the benefit of priority from Chinese Patent Application No. 2024102492324, filed on Mar. 5, 2024, which is incorporated by reference herein.

### TECHNICAL FIELD

The present disclosure relates to the field of medical equipment, and in particular, to a convenient injector for delivering artificial or allogeneic bones/grafts/scaffolds during orthopaedic operations.

### BACKGROUND

In the early stages, aseptic necrosis of the femoral head is usually treated with hip preservation surgery, which involves removing the necrotic bone tissue in the femoral head area by curettage and then filling the tunnel with artificial grafts/scaffolds or allogeneic bones. At present, artificial or allogeneic bone is generally injected into the lesion through an injector, but existing injectors have the following problems including but not limited to: inconvenient injection operation of artificial or allogeneic bone, and inconvenient cleaning and maintenance.

### SUMMARY

The present disclosure aims to provide a convenient bone graft injector for artificial or allogeneic bone, so as to solve one or more technical problems as mentioned above, and at least provide an alternation for optimizing the tool.

According to an embodiment of the present disclosure, a convenient bone graft injector for artificial or allogeneic bone is provided, including: a body, an injection tube, a locking device, and an injection device. The locking device is used to detachably lock one end of the injection tube to the body. The injection device includes a push rod, an injection elastic member, and a locking and releasing mechanism arranged on the body. The injection elastic member acts between one end of the push rod and the body, and the injection elastic member is configured to provide an elastic push to the push rod, such that the other end of the push rod extends into the locked end of the injection tube and moves towards the other end. The locking and releasing mechanism acts between the push rod and the body.

The beneficial effects of the present disclosure are as follows. According to the present disclosure, one end of the injection tube is locked to the body through the locking device, and the injection tube is detached from the body when it needs to be either cleaned or amended. Before the injection operation is performed, the bone marrow therapeutic fluid is pre-loaded into the injection tube. When the injection operation is performed, the other end of the injection tube is extended into the lesion, and the push rod is released by the locking and releasing mechanism. In this case, the push rod, under the action of the injection elastic member, extends into the end of the injection tube locked to the body and moves towards the other end of the injection tube, causing the bone marrow therapeutic fluid (artificial or allogeneic bone) in the injection tube to be injected into the bone. During the injection process, the push rod can be locked by the locking and releasing mechanism at any time to stop the injection. Throughout the entire injection process, the push rod can be locked and released at any time to control the speed and amount of injected reagents, making the operation convenient.

As a further improvement of the above technical scheme, the locking device includes a locking sleeve and a clamping sleeve. The injection tube is inserted into the clamping sleeve, and one end of the clamping sleeve has an outer tapered head structure, which is provided with at least one locking gap. The locking sleeve is sleeved over the clamping sleeve, and an inner periphery of the locking sleeve is provided with an inner tapered hole structure matched and fitted with the outer tapered head structure. The locking sleeve is movable along the clamping sleeve, such that the inner tapered hole structure locks and releases the outer tapered head structure.

In this scheme, the injection tube is locked by the fitting of the locking sleeve and the clamping sleeve. When the injection tube needs to be fixed, one end of the injection tube is extended into the clamping sleeve, such that the outer tapered head structure is sleeved over the injection tube, and then the locking sleeve and the clamping one move relative to each other in an axial direction, such that the inner tapered hole structure in the locking sleeve tightly holds the outer tapered head structure, and the locking gap on the outer tapered head structure is reduced to tightly hold the injection tube, thereby locking the tube. When the injection tube needs to be detached, the locking sleeve and the clamping sleeve move axially in opposite directions, such that the inner tapered hole structure in the locking sleeve releases the outer tapered head structure, thereby releasing the injection tube.

As a further improvement of the above technical scheme, one end of the locking sleeve away from the inner tapered hole structure is connected with an outer periphery of the clamping sleeve.

In this scheme, the relative movement between the locking sleeve and the clamping sleeve in the axial direction is realized by rotating the locking sleeve or the clamping sleeve. When the locking sleeve or the clamping sleeve are rotated, the locking sleeve moves along the clamping sleeve. Meanwhile, the number of relative rotations between the locking sleeve and the clamping sleeve is adjusted as required, so as to realize the adjustment of a clamping force for the injection tube. The threaded connection between the locking sleeve and the clamping one has a self-locking function.

As a further improvement of the above technical scheme, a stepped hole is formed at an outer side of the body, and a stepped joint is provided at an end of the clamping sleeve away from the outer tapered head structure and is in sleeve connection with the stepped hole.

In this scheme, the clamping sleeve is fixed on the body, and the injection tube is locked or released by rotating the locking sleeve. The clamping sleeve is in sleeve connection with the stepped hole at the outer side of the body through the stepped joint, such that quick installation can be realized.

As a further improvement of the above technical scheme, the locking and releasing mechanism includes a locking seat sleeve, at least one locking ball, and a push-out locking sleeve. The push-out locking sleeve is inserted into the locking seat sleeve, and the push-out locking sleeve and the locking seat sleeve are relatively movable along the axial direction. The push rod is slidably inserted in the push-out locking sleeve, and at least one locking and releasing through-hole is formed in an outer periphery wall of the push-out locking sleeve. The locking ball is movably provided in the locking and releasing through-hole, and a tapered hole section that narrows from one end to the other is provided on the inner periphery of the locking seat sleeve. The tapered hole section is abutted against a part of the locking ball located outside the locking and releasing through-hole, such that a part of the locking ball located inside the locking and releasing through-hole is abutted against an outer periphery of the push rod.

In this scheme, the relative position of the push-out locking sleeve and the locking seat sleeve in the axial direction is adjusted, to control the tapered hole section to push the locking ball to clamp the push rod.

As a further improvement of the above technical scheme, the locking and releasing mechanism further includes a locking elastic member, which acts axially between the push-out locking sleeve and the locking seat sleeve, such that the tapered hole section pushes the locking ball to move into the locking and releasing through-hole to clamp the push rod.

In the natural state, the tapered hole section in this scheme is kept, by the locking elastic member, at the position of pressing against the locking ball, such that the locking ball is kept in a state of clamping the push rod. In other words, in the natural state, the injector is in the working state of non-injection. When the relative position between the push-out locking sleeve and the locking seat sleeve in the axial direction is adjusted, the tapered hole section releases the locking ball, and the releasing of the push rod is realized.

As a further improvement of the above technical scheme, the locking seat sleeve is fixed in the body, and a movable cavity section is assigned in the locking seat sleeve. One end of the push-out locking sleeve is provided with a resistance ring that is slidably provided in the movable cavity section, and two ends of the locking elastic member respectively act on the resistance ring and one end of the movable cavity section away from the resistance ring.

In this scheme, the locking seat sleeve is fixed in the body to drive the locking ball to move axially along the tapered hole section. The tapered hole section is fixed, and the locking ball is moved along the axial direction.

When controlling the injection action, it may only need to control the push-out locking sleeve. Meanwhile, the locking elastic member in this scheme is assigned in the movable cavity section, thus realizing hidden installation. The locking elastic member keeps the push-out locking sleeve in a locked position through the resistance ring.

As a further improvement of the above technical scheme, the locking and releasing mechanism further includes a push-out trigger rotatably installed on the body. One end of the push-out locking sleeve away from the resistance ring is provided with a snap ring arranged at an end side of the lock seat sleeve. One end of the push-out trigger is engaged with the snap ring, and the other end of the push-out trigger extends out of the body. The body is provided with a knob defining an angle with the push-out trigger, and the push-out trigger is connected to a reset spring for resetting the push-out trigger.

In this scheme, the push-out trigger is pulled to drive the push-out locking sleeve to move, and the locking ball is driven to move towards the end of the tapered hole section with a larger diameter, then the push rod is released, and the push rod performs injection movement under the action of the injection elastic member.

As a further improvement of the above technical scheme, the push rod is sleeved with a positioning ring, which is adjustable along an axial direction of the push rod. The injection elastic member acts between the positioning ring and an inner wall of the body along the axial direction of the push rod.

In this scheme, an axial position of the positioning ring on the push rod is adjusted as required, so as to adjust an elastic force of the injection elastic member and inject a set amount of bone grafts or scaffolds.

As a further improvement of the above technical scheme, an outer periphery of the positioning ring is connected to a pull rod, and the body is provided with a pull groove extending along the axial direction of the push rod. The pull rod extends out from the pull groove, and a locking slot engaged with the pull rod is arranged at one end of the pull groove away from the injection tube.

After the push rod completes the injection, the push rod is back to its original position through the pull rod. The pull rod can be engaged with the locking slot at the end of the pull groove to lock the push rod. In this case, the push rod is not controlled by the locking and releasing mechanism to avoid inadequate movement.

### BRIEF DESCRIPTION OF DRAWINGS

The present disclosure will be further illustrated in conjunction with the accompanying drawings and embodiments, in which:

FIG. 1 is a schematic structural view of a convenient bone graft injector for artificial or allogeneic bone according to an embodiment of the present disclosure;

FIG. 2 is an exploded view of a convenient bone graft injector for artificial or allogeneic bone according to an embodiment of the present disclosure;

FIG. 3 is a front sectional view of a convenient bone graft injector for artificial or allogeneic bone with a push rod in a retracted state according to an embodiment of the present disclosure;

FIG. 4 is an enlarged view of part A in FIG. 3; and

FIG. 5 is a front sectional view of a convenient bone graft injector for artificial or allogeneic bones/scaffolds with a push rod in a pushed-out state according to an embodiment of the present disclosure.

### DETAILED DESCRIPTION

This section will give a detailed description of specific embodiments of the present disclosure. Preferred embodiments of the present disclosure are shown in the accompanying drawings. The function of the accompanying drawings is to supplement the description of the text part of the description, such that those having ordinary skills in the art can intuitively and vividly understand each technical feature and the overall technical scheme of the present disclosure, but the accompanying drawings are not intended to limit the scope of protection of the present disclosure.

In the description of the present disclosure, it should be understood that for the description of orientations, the orientation or positional relationships indicated by the terms such as “upper”, “lower”, “front”, “rear”, “left”, “right”, etc., are based on orientation or positional relationships shown in the accompanying drawings, and are used only for ease and brevity of illustration and description, rather than indicating or implying that the mentioned apparatus or element must have a particular orientation or must be constructed and operated in a particular orientation. Therefore, such terms should not be construed as limiting of the present disclosure.

It should be understood that in the description of the present disclosure, the term “at least one” means one or more, the term “a plurality of” (or multiple) means two or more, the term such as “greater than”, “less than”, “exceed” or variants thereof prior to a number or series of numbers is understood to not include the number adjacent to the term. The term “at least” prior to a number or series of numbers is understood to include the number adjacent to the term “at least”, and all subsequent numbers or integers that could logically be included, as clear from context.

In the description of the present disclosure, unless otherwise explicitly defined, the terms such as “provide”, “install/mount” and “connect” should be understood in a broad sense, and those having ordinary skills in the art can reasonably determine the specific meanings of the above terms in the present disclosure based on the specific contents of the technical scheme.

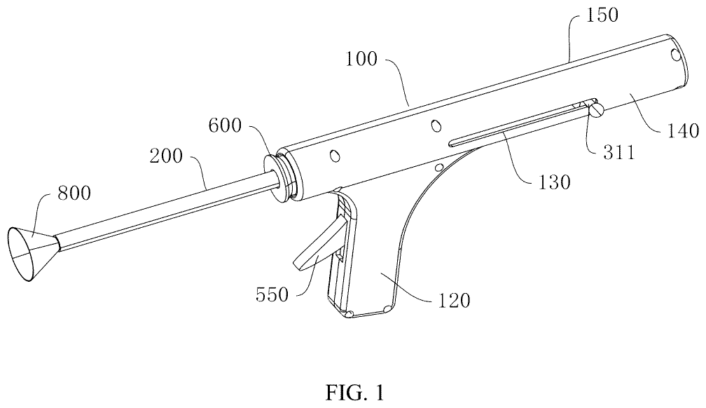

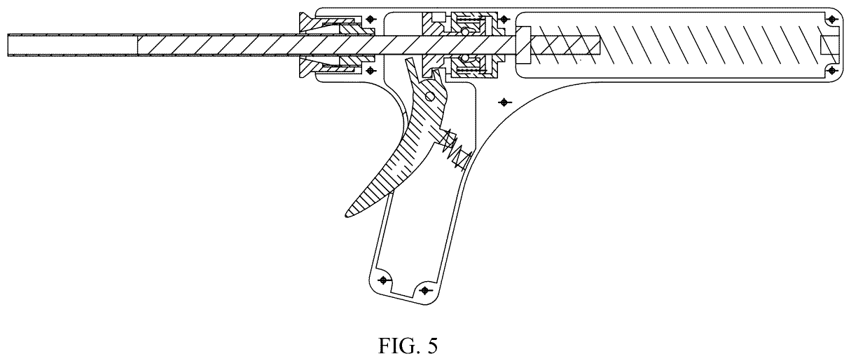

Referring to FIG. 1 to FIG. 5, in an embodiment of the present disclosure, a convenient bone graft injector for artificial or allogeneic bone is implemented as follows.

The convenient bone graft injector for artificial or allogeneic bone in this embodiment includes a body 100, an injection tube 200, a locking device, and an injection device.

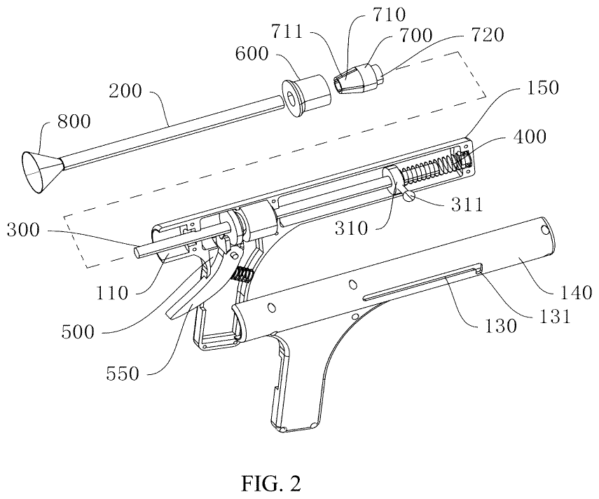

As shown in FIG. 1 and FIG. 2, the body 100 includes a front housing 140 and a rear housing 150 fitted together, which is convenient to install the locking device and injection device on the body 100, and the left end of the body 100 is an injection installation end.

In this embodiment, the locking device is arranged at the left end of the body 100. The injection tube 200 has a hollow tubular structure, and the right end of the injection tube 200 is connected to the locking device. The locking device is used to detachably lock the right end of the injection tube 200 to the left end of the body 100, and the injection tube 200 is detached from the body 100 when cleaning and maintenance of the injection tube 200 is required.

In this embodiment, the locking device includes a locking sleeve 600 and a clamping sleeve 700 which are both hollow. The right end of the injection tube 200 is inserted into the clamping sleeve 700, and a left end of the clamping sleeve 700 has an outer tapered head structure 710. The outer tapered head structure 710 has an outer diameter increasing progressively from left to right. An outer periphery of the outer tapered head structure 710 is provided with at least one locking gap 711, which extends axially along the clamping sleeve 700. In this embodiment, there are a plurality of locking gaps 711, which are arranged at annular intervals, such that a locking block is formed between two adjacent locking gaps 711.

The locking sleeve 600 is sleeved over the clamping sleeve 700 from left to right. An inner periphery of the left end of the locking sleeve 600 is provided with an inner tapered hole structure 610 matched and fitted with the outer tapered head structure 710. The inner tapered hole structure 610 has an inner diameter increasing progressively from left to right, and an inner tapered surface of the inner tapered hole structure 610 has the same taper as an outer tapered surface of the outer tapered head structure 710.

In this embodiment, the locking sleeve 600 is movable along an axis of the clamping sleeve 700, such that the inner tapered hole structure 610 locks and releases the outer tapered head structure 710. In this embodiment, the injection tube 200 is locked by the fitting of the locking sleeve 600 and the clamping sleeve 700. When the injection tube 200 needs to be fixed, the right end of the injection tube 200 is extended into the clamping sleeve 700, such that the outer tapered head structure 710 is sleeved over the injection tube 200, and then the locking sleeve 600 and the clamping sleeve 700 move relative to each other in an axial direction. In this embodiment, the locking sleeve 600 moves to the right relative to the clamping sleeve 700, such that the inner tapered hole structure 610 in the locking sleeve 600 tightly holds the outer tapered head structure 710, and the locking gap 711 on the outer tapered head structure 710 is reduced to tightly hold the injection tube 200, thereby achieving the locking of the injection tube 200. When the injection tube 200 needs to be detached, the locking sleeve 600 and the clamping sleeve 700 move in opposite directions in the axial direction, such that the inner tapered hole structure 610 in the locking sleeve 600 releases the outer tapered head structure 710, thereby achieving the releasing of the injection tube 200 and the detaching of the injection tube 200.

Regarding the relative movement between the locking sleeve 600 and the clamping sleeve 700, in this embodiment, an inner periphery of a right end of the locking sleeve 600 is in threaded connection with an outer periphery of a right end of the clamping sleeve 700, and the right end of the clamping sleeve 700 is fixedly connected to the body 100.

In this embodiment, the relative movement between the locking sleeve 600 and the clamping sleeve 700 in the axial direction is realized by rotating the locking sleeve 600. When the locking sleeve 600 is rotated, the locking sleeve 600 moves along the clamping sleeve 700, and the number of relative rotations between the locking sleeve 600 and the clamping sleeve 700 is adjusted according to needs, so as to realize the adjustment of a clamping force for the injection tube 200. The threaded connection between the locking sleeve 600 and the clamping sleeve 700 has a self-locking function.

In this embodiment, a stepped hole 110 is formed at the left end of the body 100, and a stepped joint 720 is provided at the right end of the clamping sleeve 700 and is inserted in the stepped hole 110. The clamping sleeve 700 is in sleeve connection with the stepped hole 110 at an outer side of the body 100 through the stepped joint 720, such that quick installation can be realized.

In some other embodiments, positioning adjustment between the locking sleeve 600 and the clamping sleeve 700 can be achieved by means of a pin and a pin hole.

In addition, in some other embodiments, the locking sleeve 600 is fixedly connected to the body 100, while the clamping sleeve 700 is not connected to the body 100.

The injection device includes a push rod 300, an injection elastic member 400, and a locking and releasing mechanism 500 arranged in the body 100. The push rod 300 also extends left and right, and a left end of the push rod 300 extends into the right end of the injection tube 200. The injection elastic member 400 acts between the right end of the push rod 300 and the body 100.

In this embodiment, the injection elastic member 400 is configured to provide an elastic push towards the left side to the push rod 300, such that the left end of the push rod 300 extends into the right end of the injection tube 200 and moves towards the left end within the injection tube 200.

The locking and releasing mechanism 500 is connected between the push rod 300 and the body 100, and is configured to lock and release the push rod 300.

Before the injection operation is performed, a bone marrow therapeutic fluid (artificial or allogeneic bone) is pre-loaded into the injection tube 200. When the injection operation is performed, the left end of the injection tube 200 is extended into the lesion, and the push rod 300 is released by the locking and releasing mechanism 500. In this case, the push rod 300, under the action of the injection elastic member 400, extends into the right end of the injection tube 200 and moves towards the left end of the injection tube 200, such that the bone marrow therapeutic fluid (artificial or allogeneic bone) in the injection tube 200 can be injected into the bone. During the injection process, the push rod 300 can be locked by the locking and releasing mechanism 500 at any time to stop the injection. Throughout the entire injection process, the push rod 300 can be locked and released by the locking and releasing mechanism 500 at any time to control the pace and amount of injection, making the operation convenient.

The locking and releasing mechanism 500 includes a locking seat sleeve 510, at least one locking ball 520, and a push-out locking sleeve 530. Both the push-out locking sleeve 530 and the locking seat sleeve 510 are hollow.

In this embodiment, the push-out locking sleeve 530 is inserted into the locking seat sleeve 510, and the push-out locking sleeve 530 and the locking seat sleeve 510 are relatively movable in a left-right direction. The push rod 300 slides through the push-out locking sleeve 530.

At least one locking and releasing through-hole 531 is provided in an outer periphery wall of the push-out locking sleeve 530, and the number of locking and releasing through-holes 531 depends on the number of locking balls 520. In this embodiment, there are three locking balls 520 and three locking and releasing through-holes 531, and the three locking and releasing through-holes 531 are arranged in the outer periphery wall of the push-out locking sleeve 530 at annular intervals. The locking balls 520 are movably provided in the locking and releasing through-holes 531 in one-to-one correspondence.

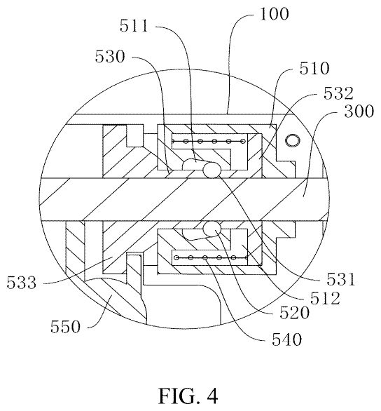

A tapered hole section 511 that narrows from one end to the other is provided on the inner periphery of the locking seat sleeve 510. As shown in FIG. 4, the tapered hole section 511 in this embodiment has a diameter decreasing progressively from left to right, and the inner tapered surface of the tapered hole section 511 is abutted against a part of the locking ball 520 located outside the locking and releasing through-hole 531, such that a part of the locking ball 520 located inside the locking and releasing through-hole 531 is abutted against an outer periphery of the push rod 300. In this embodiment, the relative position of the push-out locking sleeve 530 and the locking seat sleeve 510 in the axial direction is adjusted, to control the tapered hole section 511 to push the locking ball 520 to clamp the push rod 300.

Furthermore, the locking and releasing mechanism 500 further includes a locking elastic member 540, which acts between the push-out locking sleeve 530 and the locking seat sleeve 510 along the left and right direction, i.e., the axial direction, such that the tapered hole section 511 pushes the locking ball 520 to move into the locking and releasing through-hole 531 to clamp the push rod 300.

In the natural state, the tapered hole section 511 is kept, by the locking elastic member 540, at the position of pressing against the locking ball 520, such that the locking ball 520 is kept in a state of clamping the push rod 300. In other words, in the natural state, the injector is in the working state of non-injection. When the relative position between the push-out locking sleeve 530 and the locking seat sleeve 510 in the axial direction is adjusted, the tapered hole section 511 releases the locking ball 520, and the releasing of the push rod 300 is realized.

In this embodiment, the locking seat sleeve 510 is fixed in the body 100, and a movable cavity section 512 is arranged in the locking seat sleeve 510. A right end of the push-out locking sleeve 530 is provided with a resistance ring 532 that is slidably provided in the movable cavity section 512. Left and right ends of the locking elastic member 540 respectively act on a left end of the movable cavity section 512 and the resistance ring 532. In this embodiment, the tapered hole section 511 is fixed, and the locking ball 520 is moved along the axial direction.

When controlling the injection action, it may only need to control the push-out locking sleeve 530. Meanwhile, the locking elastic member 540 in this scheme is arranged in the movable cavity section 512, thus realizing hidden installation. The locking elastic member 540 keeps the push-out locking sleeve 530 in a locked position through the resistance ring 532.

In this embodiment, the locking and releasing mechanism 500 further includes a push-out trigger 550 rotatably installed on the body 100. A left end of the push-out locking sleeve 530 is provided with a snap ring 533 arranged at an end side of the locking seat sleeve 510. One end of the push-out trigger 550 is engaged with the snap ring 533, and the other end of the push-out trigger 550 extends out of the body 100. The body 100 is provided with a knob 120 defining an angle with the push-out trigger 550. The push-out trigger 550 is connected to a reset spring 551 for resetting the push-out trigger 550. By pulling the push-out trigger 550, the push-out locking sleeve 530 is moved, and the locking ball 520 is driven to move to the end of the tapered hole section 511 with a larger diameter, then the push rod 300 is released, and the push rod 300 performs injection movement under the action of the injection elastic member 400.

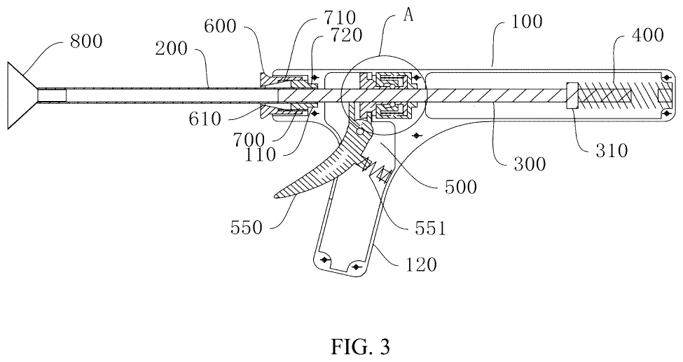

Specifically, as shown in FIG. 3, when no operation is performed, an upper end of the push-out trigger 550 moves to the right under the action of the reset spring 551, and the push-out locking sleeve 530 moves to the right under the action of the locking elastic member 540, which drives the locking ball 520 to move to the right, such that the locking ball 520 is located at a small tapered surface edge of the tapered hole section 511, and the locking ball 520 locks the push rod 300 to stop the injection.

When the push-out trigger 550 is pulled, the upper end of the push-out trigger 550 moves to the left, and the push-out trigger 550 drives the push-out locking sleeve 530 to move to the left, to drive the locking ball 520 to move to the left, such that the locking ball 520 is located at a large tapered surface edge of the tapered hole section 511, and the push rod 300 is pushed to the left under the action of the injection elastic member 400. During the whole operation, the push-out trigger 550 can be easily pulled or released at any time to control the pace and amount of injection, making the operation convenient.

Furthermore, the push rod 300 is sleeved with a positioning ring 310, which is adjustable along the axial direction of the push rod 300. The injection elastic member 400 acts between the positioning ring 310 and an inner wall of the body 100 along the axial direction of the push rod 300.

An axial position of the positioning ring 310 on the push rod 300 is adjusted according to actual needs, so as to adjust an elastic force of the injection elastic member 400 and adjust an injection amount of artificial or allogeneic bone.

In addition, an outer periphery of the positioning ring 310 is connected to a pull rod 311, and the body 100 is provided with a pull groove 130 extending along the axial direction of the push rod 300. The pull rod 311 extends out from the pull groove 130, and a locking slot 131 engaged with the pull rod 311 is arranged at one end of the pull groove 130 away from the injection tube 200.

After the push rod completes the injection movement, the push rod 300 is pulled back to its original position through the pull rod 311. The pull rod 311 can be engaged with the locking slot 131 at the end of the pull groove 130 to lock the push rod 300. In this case, the push rod 300 is not controlled by the locking and releasing mechanism 500 to avoid wrong movement.

In this embodiment, a filler funnel 800 is further included, which is used to pre-load the bone marrow therapeutic fluid (artificial bone or allogeneic bone) into the injection tube 200, and the filler funnel is removed after the pre-loading.

In this embodiment, the injection elastic member 400 and the locking elastic member 540 are both springs, and the locking ball 520 is a steel ball.

The preferred embodiments of the present disclosure have been described in detail above, but the present disclosure is not limited to these embodiments. Those having ordinary skills in the art may also make various equivalent modifications or substitutions without departing from the protection scope of the present disclosure. These equivalent modifications or substitutions are all included within the scope defined by the claims of the present application.

What is claimed is:

1. A convenient bone graft injector for artificial or allogeneic bone, comprising:
a body;
an injection tube;
a locking device for detachably locking one end of the injection tube to the body; and
an injection device comprising a push rod, an injection elastic member, and a locking and releasing mechanism arranged on the body, wherein the injection elastic member acts between one end of the push rod and the body, and the injection elastic member is configured to provide an elastic push to the push rod, such that the other end of the push rod extends into the end of the injection tube locked to the body and moves towards the other end of the injection tube; the locking and releasing mechanism acts between the push rod and the body, and the locking and releasing mechanism is configured to lock and release the push rod;
wherein the locking and releasing mechanism comprises a locking seat sleeve, at least one locking ball, and a push-out locking sleeve, the push-out locking sleeve is inserted into the locking seat sleeve, and the push-out locking sleeve and the locking seat sleeve are relatively movable along an axial direction; the push rod is slidably inserted in the push-out locking sleeve; at least one locking and releasing through-hole is formed in an outer periphery wall of the push-out locking sleeve, and the locking ball is movably provided in the locking and releasing through-hole; a tapered hole section that narrows from one end to the other is provided on an inner periphery of the locking seat sleeve, and is abutted against a part of the locking ball located outside the locking and releasing through-hole, such that a part of the locking ball located inside the locking and releasing through-hole is abutted against an outer periphery of the push rod;
the locking and releasing mechanism further comprises a locking elastic member, which acts axially between the push-out locking sleeve and the locking seat sleeve, such that the tapered hole section pushes the locking ball to move into the locking and releasing through-hole to clamp the push rod;
the locking seat sleeve is fixed in the body, and a movable cavity section is arranged in the locking seat sleeve; one end of the push-out locking sleeve is provided with a resistance ring that is slidably provided in the movable cavity section, and two ends of the locking elastic member respectively act on the resistance ring and one end of the movable cavity section away from the resistance ring;
the locking and releasing mechanism further comprises a push-out trigger rotatably installed on the body, one end of the push-out locking sleeve away from the resistance ring is provided with a snap ring arranged at an end side of the locking seat sleeve; one end of the push-out trigger is engaged with the snap ring, and the other end of the push-out trigger extends out of the body; the body is provided with a knob defining an angle with the push-out trigger; and the push-out trigger is connected to a reset spring for resetting the push-out trigger;
the push rod is sleeved with a positioning ring, which is adjustable along an axial direction of the push rod, and the injection elastic member acts between the positioning ring and an inner wall of the body along the axial direction of the push rod; and
an outer periphery of the positioning ring is connected to a pull rod; the body is provided with a pull groove extending along the axial direction of the push rod; the pull rod extends out from the pull groove, and a locking slot engaged with the pull rod is arranged at one end of the pull groove away from the injection tube.

2. The convenient bone graft injector for artificial or allogeneic bone of claim 1, wherein:
the locking device comprises a locking sleeve and a clamping sleeve, wherein the injection tube is inserted into the clamping sleeve, and one end of the clamping sleeve comprises an outer tapered head structure which is provided with at least one locking gap; the locking sleeve is sleeved over the clamping sleeve, and an inner periphery of the locking sleeve is provided with an inner tapered hole structure matched and fitted with the outer tapered head structure; and the locking sleeve is movable along the clamping sleeve, such that the inner tapered hole structure locks and releases the outer tapered head structure.

3. The convenient bone graft injector for artificial or allogeneic bone of claim 2, wherein:
one end of the locking sleeve away from the inner tapered hole structure is in threaded connection with an outer periphery of the clamping sleeve.

4. The convenient bone graft injector for artificial or allogeneic bone of claim 3, wherein:
a stepped hole is formed at an outer side of the body, and a stepped joint is provided at one end of the clamping sleeve away from the outer tapered head structure and is in sleeve connection with the stepped hole.
